(19) United States
(12) Patent Application Publication
(10) Pub. No.: US 2002/0172732 A1
(43) Pub. Date: Nov. 21, 2002

(54) Composition comprising cocoa

(76) Inventors: Wies Ter Laak, Amsterdam (NL); Peter Julien Edward Verdegem, Zetten (NL); Rene John Raggers, Amsterdam (NL)

Correspondence Address: YOUNG & THOMPSON, 745 SOUTH 23RD STREET 2ND FLOOR, ARLINGTON, VA, 22202

(21) Appl. No.: 09/812,839

(22) Filed: Mar. 21, 2001

(51) Int. Cl. A61K 35/78

(52) U.S. Cl. 424/776; 424/773

(57) ABSTRACT

The invention pertains to a composition and a method for the treatment of mood disorders, in particular of treating, preventing or alleviating depression, mood disorders or insufficient mood, obesity, overweight, premenstrual syndrome, craving, carbohydrate craving, chocolate craving, menopausal complaints, erectile dysfunction and/or reduced libido, The composition contains cocoa or one or more of its pharmacologically active components, and a dopamine D2 receptor agonist.

### FIELD OF THE INVENTION

The invention concerns nutritional and pharmaceutical compositions containing cocoa components for improving mood.

### BACKGROUND OF INVENTION

Cocoa and chocolate comprise several advantageous pharmacologically active components, and have therefore, knowingly or unknowingly, been used to alleviate or treat certain disorders. There remains a vast interest for compositions which induce the pharmacological effects of cocoa or chocolate, however which do not have the adverse side effect induced by chocolate and/or cocoa or one or more of its pharmacological components. Products available within the art, which provide the advantageous effects of the pharmacological compounds within the cocoa/chocolate, appeared insufficient. Many cocoa-containing products have high fat or carbohydrate content, causing obesity and overweight. Alternatives to these products include diet and low fat products, such as low fat cocoa powder, cocoa extracts and the like.

Pharmacological compounds within cocoa or chocolate have been used in products providing appetite suppression and mood improvement.

WO98/02165 provides a method and composition for reducing appetite and carbohydrate craving using precursors for the neurotransmitters serotonin, dopamine, norepinephrine and histamine, which include the precursors tryptophan, phenylalanine, tyrosine and histidine. The precursors are combined together and with xanthines for synergistic effect permitting advantageously lower doses of the precursors. Concomitant administration of histidine with any of tryptophan, phenylalanine and tyrosine produces a potentiated effect in appetite suppression. Xanthines, including theobromine, caffeine and cocoa, act as potentiators of the precursors, individually and in combinations of precursors. Separate formulations with xanthines of tyrosine and/or phenylalanine are used conjointly with a formulation of tryptophan with xanthines, each administered separately at intervals of at least 20 minutes. Hydrolyzed protein is utilized as a natural tryptophan source for the combinations, together with an insulin producing carbohydrate to remove from the blood stream other amino acids competing for transport across the blood-brain barrier. Alternatively, unhydrolyzed protein may be administered along with a proteolytic enzyme to produce tryptophan in the gastrointestinal tract.

The composition described in WO98/02165 provides cocoa and a precursor of serotonin. Several drawbacks are associated with the use of the composition disclosed in WO98/02165. Large doses of tryptophan are known to increase prolactin levels making the combination of xanthines and tryptophan in compositions, without the presence of components capable of counteracting these adverse side effects, especially disadvantageous, specifically for subjects suffering from PMS, overweight and craving, and has several other adverse side effects such as reduced libido.

WO99/08681 provides a method and compositions for promoting the neural synthesis and release in an animal subject of the neurotransmitters acetylcholine, GABA, glutamate, norepinephrine, dopamine, aspartate, histamine and serotonin. Precursors for each of these neurotransmitters may be administered concomitantly with a xanthine and with one or more precursors for another neurotransmitter selected from precursors for the neurotransmitters histamine, glutamine and aspartate, in order to enhance release of the neurotransmitter in the subject. The xanthines include caffeine, theophylline and theobromine. This procedure for the promotion of synthesis and release of the neurotransmitters may be employed in the treatment of subjects having a neurotransmitter deficiency, including reduced neural tone and excessive neural activity.

Some drawbacks are associated with the method and composition disclosed in WO99/08681. It has not been recognized within this disclosure, nor has it been recognized in other art, that pharmacologically active compounds within cocoa, such as xanthines and biogenic amines have several adverse side effects, especially for the treatment, prevention or alleviation of craving, PMS, overweight, obesity, menopausal symptoms, reduced libido and erectile dysfunction. Although the disclosed composition comprises cocoa and a precursor of dopamine, this will not provide sufficient relief.

U.S. Pat. No. 6,174,542 provides a chocolate containing dietary, vitamin, mineral and herbal supplement, and food products containing the same, for treating, preventing, alleviating or managing symptoms associated with premenstrual syndrome (PMS) in woman. The chocolate containing supplement and food product containing the same comprises an effective amount of the following essential ingredients: kava kava and/or St. Johns wort; cayenne, ginger and ginseng; chickweed and/or buchu and/or pyridoxine (vitamin B6), wild yam, vitamin and mineral supplements. Examples of food products incorporating these essential ingredients are liquid beverages such as a shake, juice or cappuccino; solid snack foods such as hard candies, soft candies, gum, granola bars, chocolate bars, cookies, chocolate brownies, ice cream sandwiches or chocolate cake; and semi-solid sack foods such as ice cream, sorbet or yogurt. In an alternative embodiment, the supplement can be formulated into a powder, liquid, gel, paste, tablet, capsule or coated tablet form, rather than a specific food product. This composition provides pharmacological effects induced by chocolate (although also white chocolate is described as an embodiment, which does thus not provide these effects, Michener et al, 1994).

Prolactin increase is especially undesirable for females suffering from PMS. Very unfortunately, the inventors of U.S. Pat. No. 6,174,542 chose to include Kava Kava, a herb which is suggested to be a dopamine antagonist (Schelosky et al, 1995). Dopamine antagonists decrease dopamine action, causing an increase in prolactin levels. This effect is especially undesirable. Subjects suffering from PMS may even be adversely effected by the inclusion of Kava Kava, which might increase PMS related complaints since several symptoms related to PMS may be related to the over-sensitivity to prolactin (Horrobin 1983, Tarry, 1994).

Additionally, several disadvantages are attached to the use of pharmaceutical prolactin inhibitors currently available in the art, especially the inability of such compositions to provide sufficient relief for psychological symptoms e.g. psychic symptoms related to PMS.

The influence of prolactin inhibitor bromocriptine on PMS has been investigated by Meden-Vrtovec (1992). The efficacy of bromocriptine (Bromergon, Lek) was studied in a group of 21 women with premenstrual (PMS). A statistically significant improvement due to the administration of Bromergon was observed in symptoms associated with over-activeness to normal prolactin levels, i.e. abdominal tension, edema, weight gain and breast tenderness. Scores on the linear analog scale and physician's assessments differed regarding psychological symptoms. The investigators observed no difference in the presence of psychic symptoms in the treatment-free period on Bromergon therapy and during the administration of placebo. The results obtained suggest that Bromergon may be a useful agent for the treatment of somatic symptoms associated with PMS, while it seems somewhat less effective in PMS cases where psychic symptoms are the major complaint.

U.S. Pat. No. 5,872,127 deals with the role of prolactin in immunity and describes a method of treating immune disorders by administering a combination of a serotonin agonist and a dopamine agonist. It also describes the treatment of immune disorders by administering either a prolactin enhancer such as prolactin, melatonin, dopamine antagonists or serotonin agonists or a prolactin reducer, such as dopamine agonists, dopamine or bromocriptine, depending on the prolactin state of the subject.

The art still does not provide sufficient solutions which enable the advantageous use of chocolate, cocoa and/or its pharmacologically active compounds. In addition, the art does not provide compositions, which provide treatment, relief, or prevention of psychic symptoms and decrease prolactin levels, sensitivity or inhibit prolactin synthesis and/or secretion.

### SUMMARY OF INVENTION

The invention described below provides a solution to the shortcomings of the prior art described above, enabling subjects to advantageously use cocoa or one or more of its pharmacologically active components with significantly reduced adverse side effects induced by the use of cocoa or one or more of its pharmacologically active components.

It is an object of the invention to provide a composition comprising a) cocoa or one or more of its pharmacological active components and b) a dopamine D2 receptor agonist and optionally c) compositions capable of further increasing the serotonin level and/or d) a composition capable of influencing hormonal levels.

It is an object of his invention to provide the advantageous action of one or more pharmacologically active components in cocoa, especially mood improvement induced by cocoa or one or more of its pharmacologically active components, within a dopamine D2 receptor agonist, making the composition according to the invention especially useful for the treatment, prevention and alleviation of such disorders wherein an increased prolactin levels is undesirable.

It is an object of this invention to provide a composition, for the treatment, prevention or alleviation of craving, PMS, overweight, obesity, menopausal symptoms, reduced libido and erectile dysfunction and other disorders wherein mood improvement is desired and prolactin increase is undesired, e.g. where inhibition of prolactin release is desired.

It is a further object of this invention to provide a composition, comprising cocoa or one or more of its pharmacologically active components and a dopamine D2 receptor agonist, preferably of herbal origin, especially advantageous for the treatment, prevention or alleviation of premenstrual syndrome.

It is a further object of this invention to provide a composition, comprising cocoa or one or more of its pharmacologically active components and a dopamine D2 receptor agonist, for the treatment, prevention or alleviation of overweight and obesity. The composition according to the invention will provide mood improvement to a subject suffering from such disorders, while reducing the tendency of fat storage.

It is a further object of this invention to provide a composition, comprising cocoa or one or more of its pharmacologically active components and a dopamine D2 receptor agonist, for the treatment, prevention or alleviation of reduced libido and erectile dysfunction. The composition according to the invention will provide mood improvement to subject suffering from such disorders, while reducing reduction of sexual urges.

It is a further object of this invention to provide a composition which contributes to decreased craving, limits food intake, especially high carbohydrate food products, e.g. chocolate or other cocoa including products and still provide the psychopharmacological effects induced by the intake of cocoa/chocolate and preventing the adverse side effects.

### DETAILED DESCRIPTION OF EXAMPLARY EMBODIMENTS

The invention provides a composition suitable for alleviation, prevention or treatment of craving, PMS, menopausal discomfort, chocolate craving, carbohydrate craving, overweight, obesity, erectile dysfunction, reduced libido and is suitable for providing mood improvement, in particular in such case when prolactin increase is undesirable or inhibition of prolactin secretion is desirable.

Increase of serotonin levels, increase of serotonin sensitivity and decrease of serotonin conversion, especially in the brain, will improve mood, feeling of well-being etc. Such action can for example be accomplished by provision of serotonin precursors, serotonin, serotonin release enhancers, serotonin conversion inhibitors, serotonin re-uptake inhibitors etc, and can and often will result in increased brain level serotonin compared to a situation where such compositions were not provided.

Cocoa and many of its pharmacological active components can increase serotonin level compared to a situation where such compositions were not provided. Cocoa for example comprises serotonin, components capable of inhibiting monoamine oxidase (MAO), components capable of serving as a competitive substrate for MAO and xanthines (e.g. caffeine, theobromine and theophylline). Decreased conversion of serotonin via inhibition of MAO or provision of a competitive MOA substrate will result in elevated serotonin levels compared to a situation wherein such components were not provided. Xanthines (e.g. caffeine) have been shown to increase serotonin levels in vitro (Neblig et al, 1992).

In addition to the serotonin-mediated mood improving properties of many pharmacological components in cocoa, several pharmacologically active components are present in cocoa providing non-serotonin mediated mood improvement. Such components' include phenylethylamine (PEA), which may act to potentiate dopaminergic and noradrenergic neurotransmission and believed to be an important modulator of mood (Sabelli et al, 1995).

Furthermore, cocoa comprises cannabinoid-like fatty acids which are chemically and pharmacologically related to anandamide (di Tomaso et al, 1996). These cannabinoid-like fatty acids are believed to a) mimic cannabinoid ligands directly (activating cannabinoid receptors); b) indirectly (increasing anandamide levels and/or interfere with the brain's ability to hydrolyze anandamide) and subsequently induce mood improvement and/or extend the sense of well being. According to a preferred embodiment the mood improvement provided by cocoa or one or more of its pharmacologically active components is partially non-serotonin mediated, which subsequently results in a decreased prolactin secretion compared to a situation wherein such mood improvement was solely serotonin mediated. Cocoa and mixtures of pharmacological active components from cocoa are thus especially advantageously used in the composition according to the invention. According to a further preferred embodiment cocoa powder comprising components capable of increasing serotonin levels and components capable of providing non-serotonin mediated mood improvement (e.g. PEA and cannabinoid-like fatty acids) is used.

Serotonin (5-HT) increase is followed by an increase in prolactin release and subsequent increase of serum prolactin levels. Although the mechanism behind this serotonin mediated prolactin increase has not been completely elucidated, serotonergic neurons originating in the dorsal raphe and terminating in the hypothalamus stimulate the secretion of prolactin (Wurtman et al, 1995). Furthermore, 5HT-1ₐ, 5HT-2ₐ and 5HT-2_{c} serotonin receptor agonists have been shown to increase plasma prolactin in vivo (Bagdy et al, 1989; Li Q et at, 1996).

Prolactin is a 198 amino acid long peptide structurally related to growth hormone. It is secreted in pulses every 8-10 minutes by specialized cells in the anterior pituitary (lactotrophs). Increased prolactin levels have several adverse side effects. For example, increased prolactin levels can increase several symptoms related to PMS, such as breast pain, reduce libido.

A neurotransmitter involved in the regulation of prolactin secretion is dopamine. Dopamine binds to the dopamine receptors present on lactotroph cells. Dopamine will both bind to the dopamine D1 receptor and dopamine D2 receptor, providing both a prolactin secretion stimulatory effect via the dopamine D1 receptor and a prolactin secretion inhibiting effect via binding to the dopamine D2 receptor (Freeman et al, 2000). Since dopamine both stimulates prolactin secretion and inhibits prolactin secretion, providing dopamine or precursors thereof in a composition comprising cocoa and/or one or more of its pharmacological active components will be insufficient to inhibit prolactin release, and/or synthesis. Additionally, dopamine precursors are transported over the blood-brain barrier using the same carrier (neutral amino acid carrier) as tryptophan transport, the precursor for serotonin. Precursors of dopamine will thus compete with tryptophan, resulting in decreased brain serotonin levels which will subsequently have a mood-lowering effect. The combined oral supplementation of dopamine precursors and cocoa or xanthines will thus provide an insufficient mood enhancement or even decrease mood. Furthermore, administration of dopamine, dopamine precursors or precursors of neurotransmitters increasing the release or synthesis of dopamine will provide unreliable prolactin release inhibitory effect. The administration, especially oral administration of dopamine precursors, does not necessarily result in an increased dopamine synthesis, because numerous factors are involved and control this biosynthesis, for example the metabolic state of the body. Additionally, the conversion of precursors of dopamine to dopamine and subsequent dopamine receptor is binding are relatively slow compared to D2 receptor binding by components disclosed in this invention and thus dopamine precursors provide insufficient relief.

The composition according to the invention provides a composition comprising cocoa and/or one or more of its pharmacologically active components and a dopamine D2 receptor agonist which specifically inhibits the release of prolactin, for example from lactotroph cells in the anterior pituitary. A dopamine D2 receptor agonist will either provide an increased agonistic action on the dopamine D2 receptor compared to dopamine and/or a decreased agonistic action on other dopamine receptors, e.g. the dopamine D1 receptor, compared to dopamine.

The composition according to the invention is capable of improving mood of a subject and simultaneously decreasing the adverse side effects, which coincide with mood improvement induced by cocoa or one or more of its pharmacologically active components. Improvement of mood is generally desired for subjects having a lowered or insufficient mood or mood disturbance. Such includes the mood improvement of subjects without indications of mood disturbances and subjects suffering a mood disturbance, ranging from only mild indications (e.g. bad mood, mild depression or situational of reactive mood disturbances) to subjects suffering form severe or even clinically measurable mood disturbances (severe depression).

Several mood improving pharmacological active components are present in or can be isolated from cocoa. Exemplary components include xanthines, biogenic amines and cannabinoid like fatty acids. Especially xanthines can increase serotonin level compared to a situation where such compositions were not provided, resulting in a increase of prolactin release or a decreased inhibition of prolactin release.

The composition according to the invention provides mood improvement and simultaneously provides a composition, which prevents prolactin level increase or induces prolactin level decrease, thereby inhibiting the adverse side effects. Additionally, inhibition of prolactin release and/or decrease of prolactin levels can often contribute to the prevention, treatment or alleviation of disorders or diseases. The composition according to the invention is thus especially advantageously used by subjects which desire mood improvement without the undesired simulation of prolactin release.

Craving

Craving for food is often caused by mood disturbances, for example in females days or even weeks before the menses. Many theories exist regarding the ultimate effect reached by the craving for sugar or chocolate or other food product. An adverse side effect of craving is that it often results in overweight or even obesity.

The composition according to the invention provides improvement of mood by the inclusion of cocoa or one or more of its pharmacological active compounds, thereby reducing craving, at least partially through the provision of pharmacologically active components form cocoa capable of increasing brain level serotonin, for example by the xanthines preferably present within the composition according to the invention. Due to the increase in serotonin levels, prolactin release will be stimulated, causing many undesirable side effects, such as stimulation of food intake (Gerargo Gettens 1989). Such adverse action is counteracted by the presence of a dopamine D2 receptor agonist as present the composition according to the invention.

Premenstrual Syndrome and Menopause

Some major complaints of females suffering from hormonal imbalance related distress, i.e. premenstrual syndrome (PMS) or menopausal complaints, are the fluctuations in mood, bad mood, craving, desired for chocolate craving etc. As described above these complaints can be treated, prevented or alleviated by the use of the composition according to the invention, with reduced undesirable side effects.

Mood improvement, at least partially induced by elevated serotonin levels, will result in increased prolactin release. Prolactin increase is especially undesirable for subjects suffering form PMS since it can increase several PMS related complaints, such as breast pain and water retention and subsequent bloating. The invention thus provides a method for relieve and treatment of several PMS-related complaints and improve mood in females suffering from PMS without intensifying undesirable PMS symptoms.

Acute monthly cravings for chocolate amongst pre-menstrual women may be partly explained by the high magnesium content of chocolate (Kurzer et al, 1997). Magnesium deficiency exacerbates premenstrual tension. According to preferred embodiment magnesium is included in the composition according to the invention when especially designed for treatment or prevention of PMS or menopausal complaints. Magnesium supplementation improves PMS related symptoms, especially those related to mood (Facchinetti et al, 1991)

Many peri-menopausal females (e.g. pre-menopausal and post-menopausal) suffer from mood swings or depression. Also, prolactin increase is especially undesired for females in this period of life, since high prolactin values could be associated with increased risk of breast cancer (Wang et a., 1987 and 1988), with unfavourable prognosis in breast cancer (Marugo et al, 1988) or decreased bone density (Klibamnsky et al, 1980), implying increased risk for osteoporosis (Sanfilippo, 1999).

The invention provides a mood-enhancing composition in the form of cocoa or its pharmacologically active components, providing relief in case of (pre-, post-) menopausal depression or mood depression or mood fluctuation and provides a dopamine D2 receptor agonist which inhibits the increase of prolactin levels that may have adverse side effects such as increased risk of breast cancer or decrease in bone density.

According to an especially preferred embodiment, Cimicifuga racemosa or an extract thereof is used as a dopamine D2 agonist in the composition according to the invention when used for the treatment, alleviation or prevention of menopausal symptoms. The dopamine D2 receptor agonistic effect of Cimicifuga racemosa has been shown by Winterhof (2000).

Overweight and Obesity

Overweight and obesity are often the result of craving for food. Furthermore, subjects suffering from overweight or loosing weight often experience mood disturbances or have the desire to improve mood. The composition according to the invention can be used to prevent overweight by improvement of mood, suppressing or inhibition of the desire for craving (see above), with decreased side effects due to the presence of a dopamine D2 agonist. Furthermore, prolactin has been described to induce fat storage. Thus besides counteracting the adverse side effects of the serotonin mediated mood improvement, the dopamine D2 receptor agonist will further contribute to the desired weight loss by inhibiting prolactin release and subsequently suppress the induction of fat storage.

Furthermore, several pharmacologically active components present in cocoa have a thermogenic action, i.e. the metabolic status of subjects is increased, inducing weight loss. Such pharmacological components include theobromine, and caffeine. According to a preferred embodiment, the composition according to the invention, when used to prevent, treat or alleviate overweight or obesity comprises pharmacological active compounds having a thermogenic action. According to a further preferred embodiment, the thermogenic components present in cocoa are elevated in concentration compared to cocoa or chocolate. Elevated concentrations of thermogenic compound can for example be found in cocoa extracts.

Erectile Dysfunction and Reduced Libido

Libido is the drive to have a sexual activity. Prolactin is believed to influence libido. A state of hyperprolactinemia is often associated with a loss of libido both in male and in female subjects. The exact mechanism by which this occurs is not clear, but hypothesized causes include a reduction of pituitary gonadotropin secretion leading to reduced levels of testosterone, a reduction of the dopaminergic tone, and a direct action of prolactin on the hypothalamus and other brain areas. Experimental data in male rats suggest that high levels of prolactin inhibit sexual behavior. The parameters affected include those considered to reflect motivation (mount latency) as well as those that reflect performance (intromission latency and rate) (Doherty et al, 1981). Inhibition of sexual behavior and performance due to increased prolactin levels is especially apparent when prolactin levels have been elevated for a medium period of time (Cruz-Casallas et al, 1999).

The invention provides a mood-enhancing composition in the form of cocoa one or more of its pharmacological active components providing a good mood for sexual activities, and inhibits the increase of prolactin levels, which negatively influences the sexual desire. According to a preferred embodiment, the composition according to the invention provides cocoa or one or more of its pharmacological active components, at least comprising an effective amount of phenylethylamine. According to an especially preferred embodiment the amount of phenylethylamine present in cocoa one or more of its pharmacological components is elevated compared to chocolate.

Cocoa

The term cocoa, used within the context of this invention, includes all such compositions having a significant content of pharmacologically active components present in Theobroma cacao or fermented compositions thereof. Pharmacologically active components from cocoa comprise those components found in cocoa or chocolate, and include for example, but not limited to, endogenous cocoa amino acids mixtures, xanthines (e.g. theobromine, caffeine, theophylline), cannabinoid like fatty acids, biogenic amines (e.g. phenylethylamine, tryptamine, tyramine), epiniphrine, norephiniphrine, syneprine, minerals (e,g. magnesium) and mixtures thereof.

The cocoa or its pharmacological active components may be derived from processed or unprocessed cocoa bean (Theobroma cacao). Preferably the cocoa or one or more of its pharmacological active components comprises or is derived from fermented and subsequently heat-treated cocoa bean. According to a preferred embodiment, the cocoa or one or more of its pharmacological active compounds has elevated concentrations of one or more pharmacological active components compared to chocolate. The composition according to the invention can be used to provide specific action by increasing the weight percentage of one or more desired pharmacologically active components. According to a preferred embodiment mixtures of pharmacological active component isolated from cocoa are used, such as provided in cocoa powder or extract of cocoa.

Cocoa extracts are obtained from cocoa beans or, preferably, cocoa powder by conventional exaction, involving (hot) water extraction, alcohol extraction or extraction using chlorinated hydrocarbons, ketones, esters or other organic solvent. Also, supercritical carbon dioxide may be used as an extracting agent. The preferred extracting agent is water, alcohol or water/alcohol.

According to a preferred embodiment, low fat cocoa product, e.g. cocoa extract, having a significant content of pharmacologically active components is used. Low fat inclusion will be especially advantageous to subjects suffering form overweight obesity, subjects with the desire to prevent weight increase and subjects suffering from craving. According to an additionally preferred embodiment, the cocoa has a low carbohydrate content compared to for example chocolate, since carbohydrate will have adverse side effects such as overweight and obesity.

Preferably, the composition providing cocoa or one or more of its pharmacological components provides a name of pharmacologically active components. Exemplary and preferred weight percentages of pharmacologically active components within a composition providing cocoa or one or more of its pharmacological compounds are described in table 1.

TABLE 1

| Family | Pharmacologically active | Preferred ppm wt. | Most preferred |
|---|---|---|---|
| name | compound | (μg/g) | ppm wt. (μg/g) |
| biogenic | Phenylethylamine | ≧10 | ≧100 |
| amines | Tryptamine | ≧1 | |
| | Tyramine | ≧0.5 | ≧10 |
| xanthines | Theobromine | ≧10,000 | ≧40,000 |
| | Caffeine | ≧1,000 | ≧6,000 |
| | Theophylline | | ≧1,00 |

Preferred weight percentage (based on dry weight of the cocoa or one or more of its pharmacological active components) of pharmacologically active components in the composition providing cocoa or one or more of its pharmacological active compounds

According to a preferred embodiment the cocoa or one or more of its pharmacological active components at least comprises one or more components selected from the group of xanthines, cannabinoid-like fatty acids, biogenic amines, preferably one or more compounds selected from the group of caffeine, theobromine, theophylline, phenylethylamine, tyramine and tryptamine.

Cocoa or one or more of its pharmacologically active components can be used in a quantity of about 1 mg-250 gram per dose of the composition according to the invention, greatly depending on the weight percentage of pharmacological active component in such composition. Preferably cocoa powder or cocoa extract is used in a quantity of about 1 mg-10 g per dose, more preferably about 10 mg-5 g, most preferably about 50 mg-2 g.

Dopamine D2 Receptor Agonist

Dopamine D2 receptor mediated control of prolactin secretion is strongly desired over control of prolactin levels via increase of dopamine levels, e.g. by administration of dopamine precursors, precursors of neurotransmitters stimulating dopamine release and/or synthesis, since it will provide specific control of prolactin release. The composition according to the invention comprises a dopamine D2 receptor agonist, which interacts with the dopamine D2 receptor or lactotrophe D2 receptor, thereby inhibiting prolactin secretion.

Exemplary dopamine D2 receptor agonists include drugs such as bromocriptine and semisynthetic drugs like sclarcol glycol. Major disadvantages of these potent chemical pharmaceuticals (e.g. bromocriptine) are reported in the side effects experienced such as stomach and intestinal upset, nausea and vomiting, dizziness, headache, and fatigue. The incidence of side effects while taking bromocriptine is high.

According to a preferred embodiment, dopamine D2 receptor agonist is used displaying limited side effects. According to a further preferred an effective amount of labdane diterpenoids is used as a dopamine D2 receptor agonist. Preferred labdane diterpenoids comprise songorine, rotundifuran and 6β,7β-diacetoxy-13-hydroxy-labda-8,14-diene and labdane diterpenoids derivable from Leonurus hetereophyllus. Especially preferred labdane diterpenoids include rotundifuran and 6β,7β-diacetoxy-13-hydroxy-labda-8,14-diene.

According to a further preferred embodiment, a plant derived dopamine D2 receptor agonist is used, preferably herbal D2 receptor agonist. Preferred herbal sources of dopamine D2 receptor agonists include Leonurus hetereophyllus, Vitus agnus castus, Aconitum spp. and Cimicifuga racemosa. According to an especially preferred embodiment, herbal extracts, tinctures or fractions thereof are used to provide the dopamine D2 receptor agonist, preferably extract including labdane diterpenoid, most preferably comprising rotundifuran and 6β,7β-diacetoxy-13-hydroxy-labda-8,14-diene. Herbal dopamine D2 receptor agonists are preferably used in a quantity of about 1 mg-20 g per dose of the composition of the invention, greatly depending on the herbal composition used. Extracts of herbal compositions are preferably used in a quantity of about 1 mg to 2000 mg per dose, more preferably about 5-250 mg, most preferably about 10-100 mg.

According to an especially preferred embodiment Vitex agnus castus is used as a source for dopamine D2 receptor agonist. Extracts from Vitex agnus castus have been shown to significantly inhibit basal as well as thyrotropin-releasing hormone (THR) stimulated prolactin secretion of rat pituitary cells in vitro (Sliutz et al 1993). Furthermore the efficacy of Vitex agnus castus was investigated in a randomized double blind study vs. placebo by Milewicz et al., 1999. Aim of the study was to prove whether the elevated pituitary prolactin reserve can be reduced and deficits in luteal phase length can be normalized. The prolactin release was reduced after 3 months of treatment.

Preferably extract of Vitex agnus castus are used, enriched in dopamine D2 receptor agonists compared to Vitex agnus castus fruit. Vitex agnus castus extract can be prepared as described in Hoberg et al, 1999, According to a further preferred embodiment Vitex agnus castus extract is prepared by water-alcohol extraction, e.g. water-ethanol or water-methanol extraction. Preferably about 10-90 wt. % ethanol is used in the ethanol-water extraction solvent, more preferably about 30-80 wt. %, most preferably 50 -70 wt. %, for example 60 wt. %.

Vitex agnus castus is preferably used in a quantity of about 1 mg-20 gram per doses of the composition according to the invention, greatly depending on the Vitex agnus castus derived composition used. Extracts of Vitex agnus castus are preferably used in a quantity of about 1 mg-2000 mg per doses, more preferably about 5-250 mg, most preferably about 10-100 mg, for example 40 mg. The preferred weight ratio, based on total dry weight of the composition, between cocoa or its active components, and dopamine D2 receptor agonist is between 100:1 and 1:10, preferably between 10:1 and 1:5.

The composition according to the invention can further include compositions other than cocoa or cocoa derived materials capable of increasing serotonin levels. Such compositions include, but are not limited to tryptophan, tryptophan precursors or tryptophan metabolites (e.g. 5-hydroxytryptophan) or composition capable of increasing endogenous tryptophan availability. Cofactors of the enzyme aromatic acid decarboxylase, which converts dihydroxyphenylalanine to dopamine and 5-hydroxytryptophan to serotonin, can be advantageously provided in the composition according to the invention. Such cofactors include e.g. vitamin B6, zinc and magnesium.

The composition according to the invention can further advantageously include one or more compounds selected from polyunsaturated fatty acids (e.g. γ-linolenic acid), copper, zinc, vitamin B12 and tocopherol especially when used for treatment, prevention or alleviation of PMS.

The composition according to the invention further advantageously comprises a composition capable of influencing hormonal levels. Several mechanisms exist by which the hormone levels can be influenced in vivo. The hormone levels can be influenced by administration of mammalian hormones comprising compositions, the administration of plant hormones (phytohormones) and/or the administration of compositions capable of hormonal balancing. Compositions capable of influencing hormonal levels include pure hormones, phytohormones and herbal preparations capable of hormonal balancing and/or influencing hormonal levels. According to a preferred embodiment composition capable of hormonal balancing are used.

Use of compositions capable of influencing hormonal levels, preferably hormonal balancing, is especially preferred when the composition according to the invention is used as a composition to treat, prevent or alleviate PMS. Preferably the composition capable of hormone balancing influences hormonal levels indirectly, e.g. balancing via stimulation of hypophyse. The hypophyse is believed to provide an in vivo feedback, decreasing the chances for development of abnormal hormonal levels. Furthermore the composition capable of balancing of hormone levels will prevent sudden increases and decreases in hormonal levels which could result in mood changes or craving desire.

Preferably the compositions capable of influencing hormonal levels acts on progesterone and/or estrogen levels. According to an especially preferred embodiment the composition is progesteronic. Preferred composition used for restoring hormonal levels or preventing hormonal fluctuations e.g. providing hormonal balancing action include herbal preparations such as Angelica polymorpha, Vitex agnus castus, Wild Yam, including extracts, tinctures or fractions of one or more of the herbs. According to an especially preferred embodiment a herbal composition capable of providing both the hormone balancing and inhibition of prolactin release is used.

According to a further preferred embodiment Vitex agnus castus is used to provide hormonal balancing, especially extract of Vitex agnus castus prepared by water-alcohol extraction, e.g. water-ethanol or water-methanol extraction. Preferably about 10-90 wt. % ethanol is used in the ethanol-water extraction solvent more preferably about 30-80 wt. %, most preferably 50-70 wt. %, for example 60 wt. %,

The composition according to the invention is preferably administered orally, and is for example provided as a chocolate bar, bar, candy or other sweet. However, according to a more preferred embodiment, the composition according to the invention provides only a limited amount of calories, making the composition especially suitable for use in capsules, drinks, tablets and the like. According to an especially preferred embodiment the composition according to the invention is provided as nutritional supplement in a capsule or tablet or the like. Alternatively, the composition may be administered parenterally, for example transcutaneously using e.g. a transdermal pad.

### EXAMPLES

### Example 1

### Composition Providing Relief, Mood Improvement and Prevention of Lowered Mood

A capsule providing

40 mg 60 wt. % ethanol in water extract of Vitex agnus castus extract comprising effective amounts of rotundifuran and 6β,7β-diacetoxy-13-hydroxy-labda-8,14-diene (Max Zeller Sohne AG, Switzerland)

250 mg Cocoa extract (Natropp) providing 0.5 g tyramine, about 6 mg caffeine and about 0.075 mg theophylline.

### Example 2

### Composition for Treatment, Prevention and Alleviating of PMS

Capsule to be taken 1-10 times per day, providing per capsule:

| | | |
|---|---|---|
| 250 | mg | Cocoa powder |
| 100 | mg | Vitex Agnus castus extract comprising |
| | | effective amounts of rotundifuran and |
| | | 6β,7β-diacetoxy-13-hydroxy-labda-8,14-diene |
| | | (Max Zeller Sohne AG, CH) |
| 25 | mg | Vitamin B6 |
| 200 | mg | Magnesium |
| 180 | mg | Gamma linolenic acid |
| 7.5 | μg | Vitamin B12 |
| 150 | i.u. | Vitamin E |
| 15 | mg | zinc |
| 2 | mg | copper |

### Example 3

### Composition for Prevention and Treatment of Overweight and Reduced Libido

250 mg Cocoa powder comprising phenylethylamine, caffeine and theobromine

100 mg Vitex Agnus castus extract comprising effective amounts of rotundifuran and 6β,7β-diacetoxy-13-hydroxy-labda-8,14-diene (Max Zeller Sohne AG, CH)

### Example 4

### Chocolate Bar

Chocolate bar comprising

| | |
|---|---|
| 100 g | Chocolate |
| 2.5 mg | Bromocriptine |
| 50 mg | Vitamin B6 |

Chocolate, bromocriptine and vitamin B6 are severely mixed prior to manufacture of the chocolate bar.

### Example 5

### Composition for Prevention and Treatment of Menopausal Symptoms

250 mg Cocoa exact (Natropp) providing about 0.5 g tyramine, about 6 mg caffeine and about 0.075 mg theophylline.

80 mg Cimicifuga racemosa extract (ethanol extract, comprising 8 mg actein).

### REFERENCES

Bagdy G, Szemeredi K, Kanyicska B, Merphy D L. Different serotonin receptors mediate blood pressure, heart rate, plasma catecholamine, and prolactin response to m-chlorophenyl-piperazine in conscious rats, J Pharmacol Exp Ther 1989; 250: 72-78.

Cruz-Casallas P E, Nasello A G, Hucke E E T S, Felicio L L F, PNEC 1999, 24: 681-693.

di Tomaso E, Beltramo M, Piomelli D. Brain cannaboids in chocolate. Nature 1996, 382:677-678.

Doherty P C, Bartke A, Smith M S. Differential effects of bromocriptine treatment on LH release and copulatory behavior in hyperprolactinemic male rats. Horm Behav, 1981; 15:436-450.

Facchinetti F, Borella P, Sances G, Fioroni L, Nappi R E, Genazzani A R. Oral magnesium successfully relieves premenstrual mood changes. Obstet Gynecol 1991; 78:177-81 .

Gerargo Gettens T, Moore B, Stern J S, Horwitz B A. Prolactin stimulates food intake in a dose dependent manner. Am J Physiol 1989; R276-80.

Hoberg E, Orjala J, Meier B, Sticher O. Diterpenoids from the fruits of Vitex agnus-castas. Phytochemistry 1999; 52:1555-1558.

Jarry H, Leonhardt S, Gorkow C, Wuttke W. In vitro prolactin but not LH and FSH release is inhibited by compounds in extracts of Agnus castus: direct evidence for a dopaminergic principle by the dopamine receptor assay. Exp Clin Endocrinol 1994; 102:448-54.

Klibanski A, Neer R M, Beitins I Z, Ridgway E C, Zevras N T, McArthur J W. Decreased bone density in hyperprolactinemic women N. Engl. J. Med. 1980; 303:1511-4.

Kurzer M S. Women, Food, and Mood. Nutrition Reviews 1997; 55:268-76.

Li Q, Murakami S, Stall S, Levy M S, Brownfield D E, Nichols D E, van der Kar L D. Neuroendocrine pharmacology of three serotonin releasers; 1-(1,3-benzodioxol-5yl )-2-(methylamino)butane (MHDB), 5-methoxy-6methyl-2-aminoindan (MMAi), and p-methylthioamphetamine (MTA). J Pharmacol Exp Ther 1996; 279:1261-1267.

Meden-Vrtovec H. Bromocriptine (Bromergon, Lek) in the management of premenstrual syndrome, Clinical & Experimental Obstetrics & Gynecology 1992; 19(4):242-8.

Michener W, Rozin P. Pharmacological versus sensory factors in the satiation of chocolate craving. Physiol Behav 1994; 56:419-22.

Mileswicz A, Gejdel E, Sworen H, Sienkiewicz K, Jedrzejak J, Teucher T, Schunitz H. Vitex agnus castus-Extrakt zur Behandlung von Regeltempoanomalien infolge latenter Hyperprolakinämie. Ergebnisse einer randomisierten Plazebo-kontrollierten Doppelblindstudie. Arzneimittelforschung 1993; 43(7),752-6.

We claim:

1. A composition comprising:
a. cocoa or one or more of its pharmacologically active components, and
b. a dopamine D2 receptor agonists.

2. The composition according to claim 1, wherein the composition is suitable for treatment, prevention or alleviation of mood disorders or prevention of lowered mood.

3. The composition according to claim 1, wherein the weight ratio between said cocoa component and said dopamine D2 receptor agonist is between 100:1 and 1:10.

4. The composition according to claim 1, wherein the cocoa or one or more of its pharmacologically active components includes one or more components derived from cocoa selected from amino acids mixtures, xanthines, cannabinoid-like fatty acids, epinephrine, norepinephrine, biogenic amines and minerals such as magnesium and copper.

5. The composition according to claim 1, comprising cocoa powder.

6. The composition according to claim 1, comprising cocoa extract.

7. The composition according to claim 1, comprising low fat cocoa.

8. The composition according to claim 1, wherein the dopamine D2 receptor agonist comprises a labdane diterpenoid.

9. The composition according to claim 1, wherein the dopamine D2 receptor agonist is plant-derived.

10. The composition according to claim 1, comprising Vitex agnus castus, preferably an extract of Vitex agnus castus.

11. The composition according to claim 1, comprising Cimicifuga racemosa preferably at extract of Cimicifuga racemosa.

12. The composition according to claim 1, further comprising a composition capable of influencing hormonal levels.

13. The composition according to claim 12, further comprising a composition capable of hormonal balancing.

14. The composition according to claim 12, wherein the composition acts on progesterone and/or estrogenic levels during premenstrual, menstrual, postmenstrual or menopausal periods.

15. The composition according to claim 14, wherein said composition is indirectly progesteronic.

16. The composition according to claim 12, wherein said composition capable of influencing hormonal levels does not simulate prolactin release.

17. The composition according to claim 12, wherein the composition capable of influencing hormonal levels is selected from Angelica polymorpha, Vitex agnus castus and Wild Yam.

18. The composition according to claim 1, further comprising a component capable of increasing serotonin level or serotonin precursor level.

19. A nutritional supplement comprising 10 mg-250 of cocoa or one or more of its pharmacologically active components and 1 mg-2000 mg Vitex agnus castus, preferably an extract thereof.

20. The supplement according to claim 19, comprising 10 mg-5 g cocoa powder or cocoa extract.

21. The composition according to claim 19, further comprising a compound selected from vitamin B6, magnesium, polyunsaturated fatty acids, zinc, vitamin B12 and tocopherol.

22. A nutritional supplement comprising 10 mg-250g of cocoa or one or more of its pharmacologically active components and 1 mg-2000 mg Cimicifuga racemosa, preferably an extract thereof.

23. A method of treating, preventing or alleviating depression, mood disorders or insufficient mood, obesity, overweight, premenstrual syndrome, craving, carbohydrate craving, chocolate craving, menopausal complaints, erectile dysfunction and/or reduced libido, comprising administering to a subject in need of such treatment an effective amount of at least one pharmacologically active cocoa component and at least one dopamine D2 receptor agonist.

24. The method of claim 23, wherein said pharmacologically active cocoa component comprises a xanthine and said dopamine D2 receptor agonist comprises a labdane diterpenoid.

25. The method of claim 23, wherein said cocoa component and said agonist are administered orally.

26. A method of treating, preventing or alleviating menstrual symptoms, comprising administering to a subject in need of such treatment an effective amount of a composition according to claim 10.

27. A method of treating, preventing or alleviating menstrual symptoms, comprising administering to a subject in need of such treatment an effective amount of a composition according to claim 12.

28. A method of treating, preventing or alleviating menstrual symptoms, comprising administering to a subject in need of such treatment an effective amount of a composition according to claim 19.

29. A method of treating, preventing or alleviating peri-menopausal menstrual symptoms, comprising administering to a subject in need of such treatment a effective amount of a composition according to claim 11.

30. A method of treating, preventing or alleviating peri-menopausal menstrual symptoms, comprising administering to a subject in need of such treatment an effective amount of a composition according to claim 18.

31. A method of treating, preventing or alleviating peri-menopausal menstrual symptoms, comprising administering to a subject in need of such treatment an effective amount of a composition according to claim 22.
